(19) United States
(12) Patent Application Publication
(10) Pub. No.: US 2003/0026842 A1
(43) Pub. Date: Feb. 6, 2003

(54) Production of hard, dense particles

(76) Inventors: Steven J. Prestrelski, Fremont, CA (US); Terry L. Burkoth, Fremont, CA (US); Yuh-Fun Maa, Fremont, CA (US)

Correspondence Address: Sharon E. Crane, Ph.D., BURNS, DOANE, SWECKER & MATHIS, L.L.P., P.O. Box 1404, Alexandria, VA, 22313-1404, US

(21) Appl. No.: 10/164,413

(22) Filed: Jun. 10, 2002

Related U.S. Application Data
(60) Provisional application No. 60/296,987, filed on Jun. 8, 2001.

(51) Int. Cl. A61K 9/14; B02C 19/00

(52) U.S. Cl. 424/489; 241/1

(57) ABSTRACT

Hard, dense particles of a pharmaceutical agent, suitable for administration to a subject via a needleless syringe are described. The particles are prepared by a multi step process that entails forming particles of a first size where the particles are an admixture of a pharmaceutical agent and a macromolecular carrier and the admixture has a first glass transition temperature; admixing a plasticizer that lowers the first glass transition temperature of the admixture to a second glass transition temperature which is below the first glass transition temperature; maintaining the articles at a temperature above the second glass transition temperature for a time period adequate to cause the particles to shrink and/or collapse; removing the plasticizer from the particles to yield modified particles having an increased glass transition temperature; recovering the modified particles; and then storing the recovered particles.

### CROSS-REFERENCE TO RELATED APPLICATIONS

This application is related to U.S. provisional patent application serial No. 60/296,987, filed Jun. 8, 2001, from which application priority is claimed pursuant to 35 U.S.C. §119(e)(1) and which application is incorporated herein by reference in its entirety.

### FIELD OF THE INVENTION

This invention relates to processes for forming pharmaceutical agents in particulate solid form and to the particulate solid pharmaceutical agents so formed.

### BACKGROUND TO THE INVENTION

There are many known methods for forming powdered and granulated solids. These include crystallization and sedimentation processes, grinding and milling processes as well as more involved processes involving layering and coating steps. With a wide variety of process alternatives, often the characteristics desired for the solid product and the characteristics of the material being formed into the particles play a part in the particulating process design.

There are numerous powdered or granulated pharmaceuticals. The physical and chemical properties of the granulated pharmaceutical or biological often plays a part in its production. For example, if the pharmaceutical is heat sensitive, high temperature drying or grinding must be avoided.

The mode of administration of a solid pharmaceutical material can play a part in optimizing its physical properties and the optimal method for preparing it. In the case of powdered or granulated pharmaceuticals which are predissolved before use, as is the case with certain hormone preparations and certain over-the-counter remedies, a physical form which promotes rapid and residue-free dissolution is called for and the process for forming the solid product must be optimized to assure this.

The ability to deliver pharmaceutical agents into and through skin surfaces (transdermal delivery) provides many advantages over oral or parenteral delivery techniques. In particular, transdermal delivery provides a safe, convenient and noninvasive alternative to traditional administration systems, conveniently avoiding the major problems associated with oral delivery (e.g. variable rates of absorption and metabolism, gastrointestinal irritation and/or bitter or unpleasant drug tastes) or parenteral delivery (e.g. needle pain, the risk of introducing infection to treated individuals, the risk of contamination or infection of health care workers caused by accidental needle-sticks and the disposal of used needles).

However, despite its clear advantages, transdermal delivery presents a number of its own inherent logistical problems. Passive delivery through intact skin necessarily entails the transport of molecules through a number of structurally different tissues, including the stratum corneum, the viable epidermis, the papillary dermis and the capillary walls in order for the drug to gain entry into the blood or lymph system. Transdermal delivery systems must therefore be able to overcome the various resistances presented by each type of tissue.

In light of the above, a number of alternatives to passive transdermal delivery have been developed. These alternatives include the use of skin penetration enhancing agents, or “permeation enhancers,” to increase skin permeability, as well as non-chemical modes such as the use of iontophoresis, electroporation or ultrasound. However, these alternative techniques often give rise to their own unique side effects such as skin irritation or sensitization. Thus, the spectrum of agents that can be safely and effectively administered using traditional transdermal delivery methods has remained limited.

More recently, a novel transdermal drug delivery system that entails the use of a needleless syringe to fire powders (i.e., solid drug-containing particles) in controlled doses into and through intact skin has been described. In particular, commonly owned U.S. Pat. No. 5,630,796 to Bellhouse et al. describes a needleless syringe that delivers pharmaceutical particles entrained in a supersonic gas flow. The needleless syringe is used for transdermal delivery of powdered drug compounds and compositions, for delivery of genetic material into living cells (e.g., gene therapy) and for the delivery of biopharmaceuticals to skin, muscle, blood or lymph. The needleless syringe can also be used in conjunction with surgery to deliver drugs and biologics to organ surfaces, solid tumors and/or to surgical cavities (e.g., tumor beds or cavities after tumor resection). In theory, practically any pharmaceutical agent that can be prepared in a substantially solid, particulate form can be safely and easily delivered using such devices.

To enable powdered drug compositions to be effectively administered via this new needleless syringe technique, the powders should have certain physical characteristics. In particular, the size of the particles which form the powders should be controllable, preferably with a narrow size distribution. Further, the particle density should be high, the particles should be free-flowing under a dry environment and their moisture content should be low. Additional properties of the particles which are desired include a spherical shape and a smooth surface. Each of these properties is important to provide good skin penetration whilst avoiding damage to the particles themselves under the forces required for delivery via needleless syringe.

More specifically, the particles which are administered by means of a needleless syringe are subjected to two relatively violent events. First, they are rapidly accelerated through the air from rest to a velocity of several hundred meters per second. Then, upon impact with a target surface, typically skin, the particles rapidly decelerate to rest again. In order to achieve the required penetration of the cell wall or other biological membranes, the particles should thus be physically durable and not subject to undue breakdown or fragmentation. In the case of many biologically active materials such as proteins, however, conventional particle-forming techniques often yield materials which are relatively nondense and relatively frangible.

### SUMMARY OF THE INVENTION

We have now found a new method for forming highly dense, highly durable, strong particles of pharmaceutical agents. A macromolecular carrier is employed in admixture with the pharmaceutical agent. The carrier is present in a proportion sufficient to achieve a continuous carrier phase when the admixture is formed into particles. In accordance with the invention, the carrier is temporarily associated with a substance which plasticizes it and lowers its glass transition temperature. This can occur during the formation of the particles or afterwards. When the plasticizer is present, the formed particles are subjected to a temperature above the lowered glass transition temperature for a period adequate to cause the particles to shrink and/or collapse. The plasticizer is then removed so as to cause the glass transition temperature of the macromolecular binder to increase to a higher level. The densified particles are then collected and cooled to a temperature below the higher glass transition temperature of the densified particles. Accordingly, the present invention provides a process for preparing solid particles of a pharmaceutical agent, comprising the steps of:

(a) forming first particles of a first size, said first particles comprising

an admixture of a pharmaceutical agent and a macromolecular carrier, said carrier capable of existing as a solid and said admixture having a first glass transition temperature, and

admixed plasticizer, said plasticizer lowering the first glass transition temperature of the admixture to a second glass transition temperature which is below the first glass transition temperature;

(b) maintaining said first particles at a temperature above the second glass transition temperature for a time period adequate to cause the first particles to shrink and/or collapse to second particles of a second size, which second size is smaller than the first size;

(c) removing plasticizer from the second particles to yield modified second particles thereby raising the glass transition temperature from said second glass transition temperature to a third glass transition temperature which is higher than second glass transition temperature;

(d) recovering the modified second particles; and

(e) storing said modified second particles at a temperature below the third glass transition temperature as the desired solid particles of the pharmaceutical agent.

The particles of the pharmaceutical agent and macromolecular carrier may be formed first, and the plasticizer is added to the preformed particles. The process of this aspect of the invention comprises two steps of:

(a) forming an admixture comprising a pharmaceutical agent and macromolecular carrier said admixture having a first glass transition temperature;

(b) forming particles of a first size of said admixture;

(c) adding plasticizer to said particles of a first size to yield plasticized particles, said plasticizer lowering the glass transition temperature of the admixture to a second glass transition temperature below said first glass transition temperature;

(d) maintaining said plasticized particles at a temperature above said second glass transition temperature for a time period adequate to cause said plasticized particles to shrink and/or collapse to particles of a second size which second size is smaller than the first size;

(e) removing plasticizer from the particles of a second size to yield modified particles of a second size thereby raising the glass transition temperature from said second glass transition temperature to a third glass transition temperature which is higher than second glass transition temperature;

(f) recovering the modified particles of a second size; and

(g) storing said modified particles of the second size at a temperature below the third glass transition temperature as the desired solid particles of the pharmaceutical agent.

Alternatively, the particles of the pharmaceutical agent and macromolecular carrier may be formed with plasticizer present. This process comprises the steps of:

(a) forming an admixture comprising a pharmaceutical agent, a macromolecular carrier and plasticizer; said pharmaceutical agent and said macromolecular carrier having a first glass transition temperature when separately admixed; and said pharmaceutical agent, said macromolecular carrier and said plasticizer having a second glass transition temperature when together admixed, said second glass transition temperature being below said first glass transition temperature;

(b) forming first particles of a first size of said admixture;

(c) maintaining said first particles of a first size at a temperature above said second glass transition temperature for a time period adequate to cause said first particles to shrink and/or collapse to particles of a second size which second size is smaller than the first size;

(d) removing plasticizer from the particles of a second size to yield modified particles of a second size thereby raising the glass transition temperature from said second glass transition temperature to a third glass transition temperature which is higher than second glass transition temperature,

(e) recovering the modified particles of a second size, and

(f) storing said modified particles of the second size at a temperature below the third glass transition temperature as the desired solid particles of the pharmaceutical agent.

The invention further provides:

solid particles of a pharmaceutical agent obtained by, or obtainable by, any of the processes of the invention;

a dosage receptacle for a needleless syringe, said receptacle containing an effective amount of solid particles prepared by a process of the invention;

a needleless syringe which is loaded with solid particles prepared by a process of the invention;

a vaccine composition comprising a pharmaceutically acceptable carrier or diluent and solid particles prepared by a process of the invention;

a method of vaccinating a subject, which method comprises administering to the said subject an effective amount of solid particles preared by a process of the invention.

### DETAILED DESCRIPTION OF THE INVENTION

Before describing the present invention in detail, it is to be understood that this invention is not limited to particularly exemplified compositions or process parameters. It is also to be understood that the terminology used herein is for the purpose of describing particular embodiments of the invention only, and is not intended to be limiting.

All publications, patents and patent applications cited herein, whether supra or infra, are hereby incorporated by reference in their entirety.

It must be noted that, as used in this specification and the appended claims, the singular forms “a,” “an” and “the” include plural referents unless the content clearly dictates otherwise. Thus, for example, reference to “a particle” includes a mixture of two or more such particles, reference to “an excipient” includes mixtures of two or more such excipients, and the like.

A. Definitions

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed, and are intended to be defined as indicated below.

As used herein, the term “pharmaceutical” or “pharmaceutical agent” intends any compound or composition of matter which, when administered to an organism (human or animal) induces a desired pharmacologic and/or physiologic effect by local and/or systemic action. The term therefore encompasses those compounds or chemicals traditionally regarded as drugs, as well as biopharmaceuticals including molecules such as peptides, hormones, nucleic acids, gene constructs and the like. More particularly, the term “pharmaceutical” or “pharmaceutical agent” includes compounds or compositions for use in all of the major therapeutic areas including, but not limited to, anti-infectives such as antibiotics and antiviral agents; analgesics and analgesic combinations; local and general anaesthetics; anorexics; antiarthritics; antiasthmatic agents; anticonvulsants; antidepressants; antihistamines; anti-inflammatory agents; antinauseants; antineoplastics; antipruritics; antipsychotics; antipyretics; antispasmodics; cardiovascular preparations (including calcium channel blockers, beta-blockers, beta-agonists and antiarrythmics); antihypertensives; diuretics; vasodilators; central nervous system stimulants; cough and cold preparations; decongestants; diagnostics; hormones; bone growth stimulants and bone resorption inhibitors; immunosuppressives; muscle relaxants; psychostimulants; sedatives; tranquilizers; therapeutic proteins (e.g., antigens, antibodies, growth factors, cytokines, interleukins, lymphokines, interferons, enzymes, etc.), peptides and fragments thereof (whether naturally occurring, chemically synthesized or recombinantly produced); and nucleic acid molecules (polymeric forms of two or more nucleotides, either ribonucleotides (RNA) or deoxyribonucleotides (DNA) including both double- and single-stranded molecules, gene constructs, expression vectors, antisense molecules and the like).

By “antigen” is meant a molecule which contains one or more epitopes that will stimulate a host's immune system to make a cellular antigen-specific immune response or a humoral antibody response. Thus, antigens include polypeptides including antigenic protein fragments, oligosaccharides, polysaccharides and the like. Furthermore, the antigen can be derived from any known virus, bacterium, parasite, plant, protozoan or fungus, and can be a whole organism. The term also includes tumor antigens. Similarly, an oligonucleotide or polynucleotide which expresses an antigen, such as in DNA immunization applications, is also included in the definition of an antigen. Synthetic antigens are also included, for example polyepitopes, flanking epitopes and other recombinant or synthetically derived antigens (Bergmann et aL. (1993) Eur. J Immunol. 23:2777-2781; Bergmann et al. (1996) J. Immunol. 157:3242-3249; Suhrbier, A. (1997) Immunol. and Cell Biol. 75:402-408; Gardner et al. (1998) 12^{th} World AIDS Conference, Geneva, Switzerland, Jun. 28-Jul. 3, 1998).

The above pharmaceuticals or pharmaceutical agents, alone or in combination with other agents, are typically prepared as pharmaceutical compositions which can contain one or more added materials such as carriers, vehicles, and/or excipients. “Carriers,” “vehicles” and “excipients” generally refer to substantially inert materials which are nontoxic and do not interact with other components of the composition in a deleterious manner. These materials can be used to increase the amount of solids in particulate pharmaceutical compositions. Examples of suitable carriers include water, silicone, gelatin, waxes, and like materials. Examples of normally employed “excipients,” include pharmaceutical grades of carbohydrates including monosaccharides, disaccharides, cyclodextrans, and polysaccharides (e.g., dextrose, sucrose, lactose, trehalose, raffinose, mannitol, sorbitol, inositol, dextrans, and maltodextrans); starch; cellulose; salts (e.g. sodium or calcium phosphates, calcium sulfate, magnesium sulfate); citric acid; tartaric acid; glycine; polyvinylpyrrolidone (PVP); high molecular weight polyethylene glycols (PEG); Pluronics; surfactants; and combinations thereof. Generally, when carriers and/or excipients are used, they are used in amounts ranging from about 0.1 to 99 wt % of the pharmaceutical composition.

The term “powder” as used herein refers to a composition that consists of substantially solid particles that can be delivered transdermally using a needleless syringe device. The particles that make up the powder can be characterized on the basis of a number of parameters including, but not limited to, average particle size, average particle density, particle morphology (e.g. particle aerodynamic shape and particle surface characteristics) and particle penetration energy (P.E.).

The average particle size of the powders according to the present invention can vary widely and is generally from 0.1 to 250 μm, for example from 10 to 100 μm and more typically from 20 to 70 μm. The average particle size of the powder can be measured as a mass mean aerodynamic diameter (MMAD) using conventional techniques such as microscopic techniques (where particles are sized directly and individually rather than grouped statistically), absorption of gases, permeability or time of flight. If desired, automatic particle-size counters can be used (e.g. Aerosizer Counter, Coulter Counter, HIAC Counter, or Gelman Automatic Particle Counter) to ascertain the average particle size.

Actual particle density or “absolute density” can be readily ascertained using known quantification techniques such as helium pycnometry and the like. Alternatively, envelope (“tap”) density measurements can be used to assess the density of a powder according to the invention. The envelope density of a powder of the invention is generally from 0.5 to 25 g/cm³, preferably from 0.8 to 1.5 g/cm³.

Envelope density information is particularly useful in characterizing the density of objects of irregular size and shape. Envelope density is the mass of an object divided by its volume, where the volume includes that of its pores and small cavities but excludes interstitial space. A number of methods of determining envelope density are known in the art, including wax immersion, mercury displacement, water absorption and apparent specific gravity techniques. A number of suitable devices are also available for determining envelope density, for example, the GeoPyc™ Model 1360, available from the Micromeritics Instrument Corp. The difference between the absolute density and envelope density of a sample pharmaceutical composition provides information about the sample's percentage total porosity and specific pore volume.

Particle morphology, particularly the aerodynamic shape of a particle, can be readily assessed using standard light microscopy. It is preferred that the particles which make up the instant powders have a substantially spherical or at least substantially elliptical aerodynamic shape. It is also preferred that the particles have an axis ratio of 3 or less to avoid the presence of rod- or needle-shaped particles. These same microscopic techniques can also be used to assess the particle surface characteristics, e.g. the amount and extent of surface voids or degree of porosity.

Particle penetration energies can be ascertained using a number of conventional techniques, for example a metallized film P.E. test. A metallized film material (e.g. a 125 μm polyester film having a 350 Å layer of aluminum deposited on a single side) is used as a substrate into which the powder is fired from a needleless syringe (e.g. the needleless syringe described in U.S. Pat. No. 5,630,796 to Bellhouse et al.) at an initial velocity of about 100 to 3000 m/sec. The metallized film is placed, with the metal-coated side facing upwards, on a suitable surface.

A needleless syringe loaded with a powder is placed with its spacer contacting the film, and then fired. Residual powder is removed from the metallized film surface using a suitable solvent. Penetration energy is then assessed using a BioRad Model GS-700 imaging densitometer to scan the metallized film, and a personal computer with a SCSI interface and loaded with MultiAnalyst software (BioRad) and Matlab software (Release 5.1, The MathWorks, Inc.) is used to assess the densitometer reading. A program is used to process the densitometer scans made using either the transmittance or reflectance method of the densitometer. The penetration energy of the spray-coated powders should be equivalent to, or better than that of reprocessed mannitol particles of the same size (mannitol particles that are freeze-dried, compressed, ground and sieved according to the methods of commonly owned International Publication No. WO 97/48485, incorporated herein by reference).

The terms “nucleic acid molecule” and “polynucleotide” are used interchangeably herein and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term nucleic acid sequence is the alphabetical representation of a polynucleotide molecule. This alphabetical representation can be input into databases in a computer having a central processing unit and used for bioinformatics applications such as functional genomics and homology searching.

A “vector” is capable of transferring nucleic acid sequences to target cells (e.g., viral vectors, non-viral vectors, particulate carriers, and liposomes). Typically, “vector construct”, “expression vector”, and “gene transfer vector”, mean any nucleic acid construct capable of directing the expression of a gene of interest and which can transfer gene sequences to target cells. Thus, the term includes cloning and expression vehicles, as well as viral vectors. A “plasmid” is a vector in the form of an extrachromosomal genetic element.

A nucleic acid sequence which “encodes” a selected antigen is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, procaryotic or eucaryotic mRNA, genomic sequences from viral or procaryotic DNA or RNA, and even synthetic DNA sequences. A transcription termination sequence may be located 3′ to the coding sequence.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

The term “nucleic acid immunization” is used herein to refer to the introduction of a nucleic acid molecule encoding one or more selected antigens into a host cell for the in vivo expression of the antigen or antigens. The nucleic acid molecule can be introduced directly into the recipient subject by transdermal particle delivery. The molecule alternatively can be introduced ex vivo into cells which have been removed from a subject. In this latter case, cells containing the nucleic acid molecule of interest are re-introduced into the subject such that an immune response can be mounted against the antigen encoded by the nucleic acid molecule. The nucleic acid molecules used in such immunization are generally referred to herein as “nucleic acid vaccines.”

The term “solids content” indicates the amount of solids which are either dissolved or suspended in the solvent(s) used.

The term “subject” refers to any member of the subphylum cordata including, without limitation, humans and other primates including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The term does not denote a particular age. Thus, both adult and newborn individuals are intended to be covered. The methods described herein are intended for use in any of the above vertebrate species, since the immune systems of all of these vertebrates operate similarly.

The term “transdermal delivery” includes both transdermal (“percutaneous”) and transmucosal routes of administration, i.e. delivery by passage through the skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Berner (eds.), CRC Press, (1987).

B. General Methods

The invention is concerned with processes for producing powders suitable for transdermal delivery via needleless syringe. As such, the particles which make up the powdered composition must have sufficient physical strength to withstand sudden acceleration to several times the speed of sound and the impact with, and passage through, the skin and tissue.

In one aspect of the invention such particles are prepared by a process which includes the following steps:

A. Forming an admixture made up of a pharmaceutical agent, macromolecular carrier and plasticizer. The pharmaceutical agent in admixture with the carrier alone is capable of existing as a solid. This solid mixture has a first glass transition temperature. The plasticizer lowers the glass transition temperature of the admixture to a second glass transition temperature which is below the first glass transition temperature.

B. Forming particles of a first size of the admixture.

C. Maintaining the particles of a first size at a temperature above the second glass transition temperature for a time period adequate to cause the particles of a first size to shrink and/or collapse to particles of a second size, which second size is smaller than the first size.

D. Removing plasticizer from the particles of a second size to yield modified particles of a second size thereby raising the glass transition temperature from the second glass transition temperature to a third glass transition temperature which is higher than the second glass transition temperature.

E. Recovering the modified particles of a second size and storing them at a temperature below the third glass transition temperature.

In another aspect, suitable solid particles are prepared by an alternative process that has the following steps:

A. Forming an admixture made tip of a pharmaceutical agent and a macromolecular carrier, the admixture having a first glass transition temperature.

B Forming particles of a first size of the admixture.

C. Adding plasticizer to the particles of a first size to yield plasticized particles, the plasticizer lowering the glass transition temperature of the admixture to a second glass transition temperature below the first glass transition temperature.

D. Maintaining the plasticized particles at a temperature above the second glass transition temperature for a time period to cause the plasticized particles to shrink and/or collapse to particles of a second size which second size is smaller than the first size.

E. Removing plasticizer from the particles of a second size to yield modified particles of a second size, thereby raising the glass transition temperature from the second glass transition temperature to a third glass transition temperature which is higher than second glass transition temperature.

F. Recovering the modified particles of a second size and storing them at a temperature below the third glass transition temperature as the solid particulate biological.

The pharmaceutical agent used in the invention may be any small molecule drug substance, organic or inorganic chemical, vaccine, or peptide (polypeptide and/or protein) of interest. In particular embodiments, the pharmaceutical agent is a biopharmaceutical preparation of a peptide, polypeptide, protein or any other such biological molecule. Exemplary peptide and protein formulations include, without limitation, insulin; calcitonin; octreotide; endorphin; liprecin; pituitary hormones (e.g., human growth hormone and recombinant human growth hormone (hGH and rhGH), HMG, desmopressin acetate, etc); follicle luteoids; growth factors (such as growth hormone releasing factor (GHRF), somatostatin, somatotropin and platelet-derived growth factor); asparaginase; chorionic gonadotropin; corticotropin (ACTH); erythropoietin (EPO); epoprostenol (platelet aggregation inhibitor); glucagon; interferons; interleukins; menotropins (urofollitropin, which contains follicle-stimulating hormone (FSH); and luteinizing hormone (LH)); oxytocin; streptokinase; tissue plasminogen activator (TPA); urokinase; vasopressin; desmopressin; ACTH analogues; angiotensin II antagonists; antidiuretic hormone agonists; bradykinin antagonists; CD4 molecules; antibody molecules and antibody fragments (e.g., Fab, Fab₂, Fv and sFv molecules); IGF-1; neurotrophic factors; colony stimulating factors; parathyroid hormone and agonists; parathyroid hormone antagonists; prostaglandin antagonists; protein C; protein S; renin inhibitors; thrombolytics; tumor necrosis factor (TNF); vaccines (particularly peptide vaccines including subunit and synthetic peptide preparations); vasopressin antagonists analogues; and α-1 antitrypsin. Additionally, nucleic acid preparations, such as vectors or gene constructs for use in subsequent gene delivery, can be used.

Particularly suitable pharmaceutical agents for use herein are antigens. Any suitable antigen as defined herein may be employed. The antigen may be a viral antigen. The antigen may therefore be derived from members of the families Picornaviridae (e.g. polioviruses, etc.); Caliciviridae; Togaviridae (e.g. rubella virus, dengue virus, etc.); Flaviviridae; Coronaviridae; Reoviridae; Birnaviridae; Rhabodoviridae (e.g. rabies virus, etc.); Filoviridae; Paramyxoviridae (e.g. mumps virus, measles virus, respiratory syncytial virus, etc.); Orthomyxoviridae (e.g. influenza virus types A, B and C, etc.); Bunyaviridae; Arenaviridae; Retroviradae (e.g. HTLV-I; HTLV-II; HIV-1 and HIV-2); and simian immunodeficiency virus (SIV) among others.

Alternatively, viral antigens may be derived from a papillomavirus (e.g. HPV); a herpesvirus; a hepatitis virus, e.g. hepatitis A virus (HAV), hepatitis B virus (HBV), hepatitis C (HCV), the delta hepatitis virus (HDV), hepatitis E virus (HEV) or hepatitis G virus (HGV); and the tick-borne encephalitis viruses. See, e.g. Virology, 3rd Edition (W. K. Joklik ed. 1988); Fundamental Virology, 2nd Edition (B. N. Fields and D. M. Knipe, eds. 1991) for a description of these viruses.

Bacterial antigens for use in the invention can be derived from organisms that cause diphtheria, cholera, tuberculosis, tetanus, pertussis, meningitis and other pathogenic states, including Meningococcus A, B and C, Hemophilus influenza type B (HIB) and Helicobacter pylori. A combination of bacterial antigens may be provided, for example diphtheria, pertussis and tetanus antigens. Suitable pertussis antigens are pertussis toxin and/or filamentous haemagglutinin and/or pertactin, alternatively termed P69. An anti-parasitic antigen may be derived from organisms causing malaria and Lyme disease.

Antigens for use in the present invention can be produced using a variety of methods known to those of skill in the art. In particular, the antigens can be isolated directly from native sources, using standard purification techniques. Alternatively, whole killed, attenuated or inactivated bacteria, viruses, parasites or other microbes may be employed. Yet further, antigens can be produced recombinantly using known techniques. See, e.g., Sambrook, Fritsch & Maniatis, Molecular Cloning: A Laboratory Manual, Vols. I and II (D. N. Glover et. 1985).

Antigens for use herein may also be synthesised, based on described amino acid sequences, via chemical polymer syntheses such as solid phase peptide synthesis. Such methods are known to those of skill in the art. See, e.g. J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis, Springer-Verlag Berlin (1984) and E. Gross and J. Meienhofer, Eds., The Peptides: Analysis, Synthesis, Biology, supra, Vol. 1, for classical solution synthesis.

The pharmaceutical agent may alternatively be a nucleic acid molcule. The pharmaceutical agent can thus be a polynucleotide which expresses an antigen, such as in DNA immunization applications. An expression vector can thus be employed in which a nucleic acid sequence encoding a desired polypeptide such as an antigen is operably linked to a promoter.

Typically, the nucleic acid molecule comprises a therapeutically relevant nucleotide sequence for delivery to a subject. Thus, the nucleic acid molecule may comprise one or more genes encoding a protein defective or missing from a target cell genome or one or more genes that encode a non-native protein having a desired biological or therapeutic effect (e.g., an antiviral function). The molecule may comprise a sequence capable of providing immunity, for example an immunogenic sequence that serves to elicit a humoral and/or cellular response in a subject, or a sequence that corresponds to a molecule having an antisense or ribozyme function. For the treatment of genetic disorders, functional genes corresponding to genes known to be deficient in the particular disorder can be administered to a subject.

Suitable nucleic acids for delivery include those used for the treatment of inflammatory diseases, autoimmune, chronic and infectious diseases, including such disorders as AIDS, cancer, neurological diseases, cardivascular disease, hypercholestemia; various blood disorders including various anemias, thalassemia and hemophilia; genetic defects such as cystic fibrosis, Gaucher's Disease, adenosine deaminase (ADA) deficiency, emphysema, etc. A number of antisense oligonucleotides (e.g., short oligonucleotides complementary to sequences around the translational initiation site (AUG codon) of an mRNA) that are useful in anitsense therapy for cancer and for viral diseases have been described in the art. See, e.g., Han et al. (1991) Proc. Natl. Acad. Sci USA 88:4313; Uhlmann et al. (1990) Chem. Rev. 90:543, Helene et al. (1990) Biochim. Biophys. Acta. 1049:99; Agarwal et al. (1988) Proc. Natl. Acad. Sci. USA 85: 7079; and Heikkila et al. (1987) Nature 328:445. A number of ribozymes suitable for use herein have also been described. See, e.g., Chec et al. (1992) J. Biol. Chem. 267: 17479 and U.S. Pat. No. 5,225,347 to Goldberg et al.

For example, in methods for the treatment of solid tumors, genes encoding toxic peptides (i.e., chemotherapeutic agents such as ricin, diptheria toxin and cobra venom factor), tumor suppressor genes such as p53, genes coding for mRNA sequences which are antisense to transforming oncogenes, antineoplastic peptides such as tumor necrosis factor (TNF) and other cytokines, or transdominant negative mutants of transforming oncogenes, can be delivered for expression at or near the tumor site.

Similarly, nucleic acids coding for peptides known to display antiviral and/or antibacterial activity, or stimulate the host's immune system, can also be administered. The nucleic acid may encode one of the various cytokines (or functional fragments thereof), such as the interleukins, interferons, chemokines, chemotaxic factors, and colony stimulating factors. The nucleic acid may encode an antigen for the treatment or prevention of a number of conditions including but not limited to cancer, allergies, toxicity and infection by a pathogen such as, but not limited to, fungus, viruses including Human Papiloma Viruses (HPV), HIV, HSV2/HSV1, influenza virus (types A, B and C), Polio virus, RSV virus, Rhinoviruses, Rotaviruses, Hepaptitis A virus, Norwalk Virus Group, Enteroviruses, Astroviruses, Measles virus, Par Influenza virus, Mumps virus, Varicella-Zoster virus, Cytomegalovirus, Epstein-Barr virus, Adenoviruses, Rubella virus, Human T-cell Lymphoma type I virus (HTLV-1), Hepatitis B virus (HBV), Hepatitis C virus (HCV), Hepatitis D virus, Pox virus, Marburg and Ebola; bacteria including M. tuberculosis, Chlamydia, N.gonorrhoeae, Shigella, Salmonella, Vibrio Cholera, Treponema pallidua, Pseudomonas, Bordetella pertussis, Brucella, Franciscella tulorensis, Helicobacter pylori, Leptospria interrogaus, Legionella pnumophila, Yersinia pestis, Streptococcus (types A and B), Pneumococcus, Meningococcus, Hemophilus influenza (type b), Toxoplama gondic, Complybacteriosis, Moraxella catarrhalis, Donovanosis, and Actinomycosis; fungal pathogens including Candidiasis and Aspergillosis; parasitic pathogens including Taenia, Flukes, Roundworms, Amebas, Giardia species, Cryptosporidium, Schitosoma species, Pneumocystis carinii, Trichuriasis species, and Trichinella species. The nucleic acid my also be used to provide a suitable immune response against numerous veterinary diseases, such as Foot and Mouth diseases, Coronavirus, Pasteurella multocida, Helicobacter, Strongylus vulgaris, Actinobacillus pleuropneumonia, Bovine viral diarrhea virus (BVDV), Klebsiella pneumoniae, E. Coli, Bordetella pertussis, Bordetella parapertussis and brochiseptica. Thus in one aspect, the particles of the present invention may find use as a vaccine.

The invention will also find use in antisense therapy, e.g., for the delivery of oligonucleotides able to hybridize to specific complementary sequences thereby inhibiting the transcription and/or translation of these sequences. Thus DNA or RNA coding for proteins necessary for the progress of a particular disease can be targeted, thereby disrupting the disease process. Antisense therapy, and numerous oligonucleotides which are capable of binding specifically and predictably to certain nucleic acid target sequences in order to inhibit or modulate the expression of disease-causing genes are known and readily available to the skilled practitioner. Uhlmann et al. (1990) Chem Rev. 90: 543, Neckers et al. (1992) Int. Rev. Oncogenesis 3, 175; Simons et al. (1992) Nature 359, 67; Bayever et al. (1992) Antisense Res. Dev. 2: 109; Whitesell et al. (1991) Antisense Res. Dev. 1: 343; Cook et al. (1991) Anti-cancer Drug Design 6: 585; Eguchi et al. (1991) Ann. Rev. Biochem. 60: 631. Accordingly, antisense oligonucleotides capable of selectively binding to target sequences in host cells are provided herein for use in antisense therapeutics.

The antigen or other pharmaceutical agent employed in the present invention may optionally be adsorbed into an adjuvant, such as an aluminum salt adjuvant or calcium salt adjuvant. Alternatively, the antigen or other pharmaceutical agent may be used without an adjuvant. Suitable adjuvants include aluminium hydroxide, aluminum phosphate, aluminum sulfate and calcium phosphate.

The macromolecular carrier used in the invention is broadly selected from natural and synthetic polymeric materials which exhibit a glass transition temperature.

Naturally-occurring macromolecular carriers can include carbohydrates including sugars, starches, modified starches and modified starches. Suitable starches and modified starches can have a molecular weight of up to 700,000, however starches with molecular weights of about 1,000 to about 100,000 are preferred, and those with molecular weights of about 30,000 to about 40,000 are more preferable. Modified starches that are particularly useful in the invention are carboxymethyl starch and carboxyethyl starch and amino starches. The carriers can include gums such as carrageenan, agar, alginates, dextrans and the like. The carriers can also be selected from chitin, chitosan, polypeptides including materials classed as proteins and as polyamino acids, such as collagen and derivatized collagen, gelatin, and the like. Furthermore, the carrier can be a hydrogel.

Synthetic macromolecular carriers include block copolymers of natural and synthetic polymers, polyalcohols such as polyvinyl alcohol, polyacrylates and polymethacrylates and polyhydromethyl methacrylates, block copolymers of these acrylates, polyacids such as polylactic acid, polyglycolic acid, polyglycols such as polyethylene glycol and polyhydroxyl butylene, polyketals, polyanhydrides, polyorthoesters and poloxamers.

The carrier materials used in the present invention are pharmaceutically acceptable (in the case of pharmaceutical particles) and/or biologically acceptable (in the case of particles for injection into biological systems). That is, the carrier materials must be non-damaging and non-toxic in the environment of use. To this end, materials typically employed in biology and pharmacology, such as the dextrans, the gums, the starches, polyvinyl alcohol and many of the acrylates and methacrylates which are generally recognized as safe or which are otherwise approved for pharmacological uses, are preferred. Mixed carriers can be employed as well.

The macromolecular carriers employed in the invention have glass transitions. That is, they exist in a rigid glassy state below a certain temperature and exist in a more fluid state above that temperature.

The glass transition temperatures is commonly represented by the symbol T_{g}. The T_{g} for a given composition can be determined using differential scanning calorimetry (DSC). The T_{g} is observed to be the temperature at which there is a marked change in the heat capacity of the material. Additional information concerning glass transition temperatures may be found in the articles “Differential Scanning Calorimetry Analysis of Glass Transitions” by Jan P. Wolanczyk: Cryo-Letters, 10, 73-76 (1989) and “Nature of the Glass Transition and the Glassy State” by Gibbs and DiMarzio: Journal of Chemical Physics, 28, No. 3, 373-383 (March, 1958).

A given macromolecular carrier will have a characteristic glass transition temperature. Mixtures of a given macromolecular carrier with a pharmaceutical agent will also have a characteristic glass transition temperature. These characteristic glass transition temperatures of the underlying carrier and the carrier/pharmaceutical agent mixture can range up to about 200° C., but will most commonly range from about 30° C. to about 130° C., and more preferably will range from about 30° C. to about 110° C. and most preferably from about 30° C. to about 80° C.

The plasticizer employed in this invention is a material, most commonly a liquid at room temperature, which has the following properties. First, when added to the carrier/active agent mixture, the plasticizer lowers the glass transition temperature of the mixture. Second, it can be removed from the carrier/pharmaceutical agent mixture under conditions which do not damage or destroy the carrier and/or the pharmaceutical agent. Third, the plasticizer does not interact with, damage or destroy the carrier and/or the pharmaceutical agent.

Examples of representative volatile plasticizers include water, aqueous solutions such as aqueous solutions of dimethyl ether, oxyhydrocarbons such as alcohols, ketones, ethers and esters; hydrocarbons such as alkanes and the like; halohydrocarbons such as dichloromethane; tertiary amines; and supercritical carbon dioxides, and any mixtures of the foregoing.

The proportion of carrier, active material and plasticizer should be controlled. The proportion of carrier should be selected to assure that the carrier presents a substantially continuous phase, that is the carrier does not exist as a series of separate discrete particles but rather is formed into a substantially continuous web. This sets the lower proportion of carrier.

The lower proportion of the pharmaceutical agent will be very dependent upon the activity of the active agent and the size of the dose desired to be delivered to a subject.

As between these two materials the proportions can range as follows:

Carrier 99% to 10% by weight (basis binary mixture)

Active Agent 1% to 90% by weight.

Preferred proportions with most active agents are as follows:

Carrier 95% to 20% by weight

Active Agent 5% to 80% by weight.

The proportion of plasticizer present in the particles when they are treated to cause them to shrink and/or collapse may be expressed functionally—it should be adequate to lower the glass transition temperature below the heat-treating temperature, which heat-treating temperature should be below the temperature at which any significant thermal degradation of the active substance or, for that matter, the carrier occurs.

Most commonly, the amount of plasticizer should be adequate to lower the glass transition temperature by at least about 10° C., and more preferably at least about 15° C. The amount of plasticizer is preferably controlled since the amount is lowered (or the plasticizer is removed) in subsequent process steps and there is no advantage to having gross excesses of plasticizer.

As previously noted, the glass transition temperature of the mixture, with and without plasticizer, can be determined by differential scanning calorimetry or a like method. Similarly, the degradation temperature of the active agent and the carrier can be determined by simple thermal exposure/property tests.

As overall guidelines the proportion of plasticizer is typically as follows.

| | |
|---|---|
| Carrier plus Active Agent | 70% to 99% by weight |
| | (basis tertiary mixture) |
| Plasticizer | 30% to 1% by weight |
| preferably | |
| Carrier plus Active Agent | 80% to 98% |
| Plasticizer | 20% to 2%; and |
| more preferably | |
| Carrier plus Active Agent | 90% to 98%, |
| Plasticizer | 10% to 2% |

After heat-treating, the plasticizer is removed to a level to cause the glass transition temperature to rise. Again, the desired degree of removal can be determined empirically by measuring the glass transition temperature of the heat-treated particles. The degree of removal can also be based upon the characteristics of the plasticizer and its interactions with the active agent and/or the carrier. Clearly, if the plasticizer is not an approved material or is deleterious in the organism or material being treated, it must be thoroughly removed. Similarly, if the plasticizer interacts deleteriously with the active agent or carrier it must be removed to levels that this interaction is minimized.

More commonly, however, the plasticizer does not present these issues so that the removal level can be based on the increase in glass transition temperature which can be observed directly experimentally. As guidelines, typically at least about 20% and more commonly at least about 30% or 50% of the plasticizer present at the beginning of the heat treatment is subsequently removed.

The compositions can contain mixtures of two or more active agents, multiple carrier materials, multiple plasticizers and also can include other materials commonly found in biological and pharmaceutical solids such as buffers, fillers, dispersants, colorants, and the like. Suitable excipients can include free-flowing particulate solids that do not thicken or polymerize upon contact with water, which are innocuous when administered to an individual, and do not significantly interact with the pharmaceutical agent in a manner that alters its pharmaceutical activity. Examples of normally employed excipients include, but are not limited to, pharmaceutical grades of dextrose, sucrose, lactose, trehalose, mannitol, sorbitol, inositol, dextran, starch, cellulose, sodium or calcium phosphates, calcium carbonate, calcium sulfate, sodium citrate, citric acid, tartaric acid, glycine, high molecular weight polyethylene glycols (PEG), and combinations thereof. Suitable solvents include, but are not limited to, methylene chloride, acetone, methanol, ethanol, isopropanol and water. Typically, water is used as the solvent. Generally pharmaceutically acceptable salts having molarities ranging from about 1 mM to 2M can be used. Pharmaceutically acceptable salts include, for example, mineral acid salts such as hydrochlorides, hydrobromides, phosphates, sulfates, and the like; and the salts of organic acids such as acetates, propionates, malonates, benzoates, and the like. A thorough discussion of pharmaceutically acceptable excipients, vehicles and auxiliary substances is available in REMINGTON'S PHARMACEUTICAL SCIENCES (Mack Pub. Co., N.J. 1991), incorporated herein by reference.

The overall process of the invention can be viewed as a primary particle-forming operation followed by a secondary particle-treating operation.

In the primary process the carrier and the active agent are admixed and formed into particles. In certain embodiments plasticizer is present.

These two operations can be carried out in two separate stages. For example, in the first stage a mixture of carrier and active agent, in the proportions recited above can be formed into particles.

These initial particles can be formed by processes such as spray drying, fluidized bed drying, freeze drying and spray freeze-drying. The feed stocks for these processes include solutions/suspensions of the carrier and active agent. The solvent/suspending agent for these feed materials can be an aqueous or non-aqueous liquid which can suspend or dissolve the active agent and the carrier.

The proportion of carrier and active agent in the feed solution/suspension is generally selected to minimize the amount of solvent. This is because this solvent must be removed in the particle formation process and it is economically advantageous to minimize the volume of solvent being removed and recovered.

The particles of the first step are often relatively fragile and low in density. That is, they typically have bulk densities of from about 0.1 to about 0.5 g/ml, i.e. a specific gravity of from about 0.1 to about 0.5. This compares with a typical absolute specific gravity for the carrier/agent combination (full density) of from about 1.0 to about 1.5.

The conditions employed in the initial particle formation step are selected to assure the integrity of the pharmaceutical agent. Destructive temperatures are to be avoided. With these provisos, the process conditions for forming particles are conventional for the processing equipment employed. Typically the temperature employed in the first step is in the range of from about 25° C. to about 125° C. for spray drying and from about −75° C. to about 0° for spray freeze-drying.

In the second step of the two-step process, the low density particles produced in the first step are subjected to heating in the presence of plasticizer. This can be carried out by suspending the low density particles in a non-solvent fluid, adding plasticizer and heating. The non-solvent fluid can be a liquid or a gas which does not dissolve the preformed particles. Representative liquid non-solvents include hydrocarbons, both aliphatic and aromatic, fluorocarbons and the like. Plasticizer is added in an amount suitable to give rise to the proportion of plasticizer to carrier and active agent described above. The suspending of the particles in the non-solvent is carried out in a mixer, in the case of a liquid non-solvent, or in a fluidized bed in the case of a gaseous non-solvent. Suspension of the particles is used to avoid aggolmeration or fusion of particles when the composition is above the T_{g}.

The suspended particles, with the added plasticizer, are heated to a temperature above the glass transition temperature of the carrier-plasticizer combination. The temperature is maintained above the glass transition temperature for a time period adequate for the plasticized particles to shrink and/or collapse and densify. This time will range from about 30 seconds to about five hours, more preferably from about one minute to about 3 hours. Elevated pressure such as from above atmospheric pressure up to about 50 atmospheres can be applied during the densification step to increase and speed densification.

This heating is carried out with the particles in suspension. This assures uniform densification and formation of substantially spherical particles.

After the heat treating, the particles are treated to remove or reduce plasticizer and residual non-solvent (if present). Plasticizer and non-solvent liquid can be removed by mild heating, such as to a temperature below the unplasticized glass transition temperature and applying vacuum. Alternative liquid removal steps can be used, as well.

The plasticizer/non-solvent removal is continued until the glass transition temperature of the remaining temperature carrier-agent mixture has risen to above the temperature of use and storage of the particles.

The plasticizer/non-solvent removal steps can be carried out in a fluidized bed, in a tumbler or even in a tray dryer or the like with mild heat and/or vacuum being applied, such as temperatures of from about 30° C. to about 75° C. and vacuums from just below atmospheric pressure (e.g. 700 mm Hg) to as low as 1 mm Hg or lower.

In a variation of the process, the particle forming and particle shrinking steps can be carried out in a continuous single step. In the variation, the particles are formed in the presence of plasticizer and after forming are held in the presence of plasticizer at a temperature above the plasticized glass transition temperature for the period suitable to cause the particles to shrink and/or collapse, and thereby increase in particle density.

This one-step process can be very effective when the particles are formed in a spray dryer and when air is the suspending medium for the densification step and when water is the plasticizer. In this one-step process, the same proportions and conditions described for the two-step process can be employed.

The processes are commonly carried out in a batch mode. If large volumes of particles are needed, a continuous process could be provided in which the process steps are carried out in a plug flow mode in elongated processing zones.

As mentioned above, particles may be formed by spray freeze-drying. That may entail spray freeze-drying an aqueous solution or suspension which comprises a pharmaceutical agent, a macromolecular carrier and optionally a plasticizer and which has a solids content of 10% by eight or more such as 20% by weight or more.

The solids content in the solvent system may be 25% by weight or more, more preferably 28% or 30% by weight or more and particularly preferably 40% by weight or more. The solids content of the solution or dispersion may be up to 50% by weight, up to 60% by weight or even up to 70% by weight. The upper limit depends upon, for example, the particular components of the solution or dispersion and the desired characteristics of the resulting spray freeze-dried particles.

The aqueous solution or suspension for spray freeze-drying can contain excipients chosen for use in the present invention may serve specific functions such as protein stabilization or surface protection or may be used as a bulking agent or to maintain low hygroscopicity of the powders. Although the invention is not limited to the use of any specific excipients, particularly suitable excipients include saccharides, which may be amorphous or crystalline saccharides, polymers or amino acids or physiologically acceptable salts thereof. The use of these particular excipient compositions allow the particles to collapse and densify during freezing and therefore provide powders which are particularly suitable for injection via a needleless syringe. The saccharide may be a monosaccharide, disaccharide or higher oligo- or polysaccharide. The excipients may be selected from carbohydrates, sugars and sugar alcohols. Such excipients may be crystalline or amorphous, as long as the selected excipient does not adversely affect the glassy continuous phase of the carrier.

Preferably, one, two or three of these additives are present in the solution or suspension in amounts of at least 15% by weight, preferably at least 20% by weight, such as at least 25%, 28% or 30% by weight and more preferably at least 40% by weight. Such additives may be present in amounts of up to 50% by weight, up to 60% by weight or even up to 70% by weight. The upper limit depends upon, for example, the particular additives used and the desired characteristics of the resulting spray freeze-dried particles. Most preferably one or two different excipients are used.

Suitable amorphous saccharides include sugars. The amorphous excipient may thus be selected from dextrose, sucrose, lactose, trehalose, cellobiose, raffinose, isomaltose and other carbohydrates such as cyclodextrins. Such sugars are capable of stabilizing proteins used as pharmaceutical agents during the spray-freeze-drying process and during long-term storage.

Suitable crystalline carbohydrates, sugars and sugar alcohols include mannitol, sorbitol and allitol. The combination of such a crystalline excipient with an amorphous excipient, typically an amorphous sugar, encourages the collapse of particles during freeze-drying and aids the formation of dense particles.

Suitable polymers include polysaccharides such as dextran or maltodextran, starch, cellulose, polyvinylpyrrolidone (PVP), polyvinyl alcohol (PVA) and polyethylene glycols (PEG). Dextrans are preferred. Various grades of dextran are available. A suitable dextran may have a molecular weight of greater than about 15,000 such as from about 15,000 to about 45,000, from about 60,000 to about 90,000 or from about 100,000 to about 200,000. The addition of a polymer as an excipient tends to provide powders with improved flowability and may also provide increased protein stability.

Suitable amino acids and physiologically acceptable salts of amino acids include glycine, alanine, glutamine, arginine, lysine and histidine and salts thereof such as alkali or alkaline earth metals salts such as sodium, potassium or magnesium salts or salts with other amino acids such as glutamate or aspartate salts.

The most preferred combinations of excipients for use in the present invention include an amorphous saccharide with a crystalline saccharide and optionally also a polymer and/or an amino acid or a salt thereof. The excipients may comprise an amorphous saccharide which is typically present in an amount of from 10 to 90% by weight, preferably from 50 to 80% and more preferably from 60 to 75% by weight; and a crystalline saccharide which is typically present in an amount of from 10 to 90% by weight, preferably from 20 to 50% and more preferably from 25 to 40% by weight. This combination of excipients is preferably used together with a surfactant which is typically present in an amount of from 1 to 5% by weight. Alternatively, the additives may comprise an amorphous saccharide which is typically present in an amount of from 10 to 80% by weight, preferably from 20 to 50%, more preferably from 25 to 35% by weight; a crystalline saccharide which is typically present in an amount of from 10 to 80% by weight, preferably from 20 to 50%, more preferably from 25 to 35% by weight; and a polymer or an amino acid or salt thereof, each of which is present in an amount of from 10 to 80% by weight, preferably from 30 to 60%, more preferably from 30 to 50% by weight.

The most preferred additive combinations include trehalose/mannitol, typically at a weight ratio of about 70/30; trehalose/mannitol/dextran, typically at a weight ratio of about 30/30/40; trehalose/mannitol/PVP, typically at a weight ratio of about 30/30/40; or trehalose/mannitol/arginine glutamate, typically at a weight ratio of about 30/30/40. Particularly suitable particles can be prepared from an aqueous solution or dispersion of a pharmaceutical agent which further comprises trehalose, mannitol and dextran in a weight ratio of from about 3:3.:4 to about 4:4:3.

Whilst the excipient combinations described above are not essential for use in the present invention, they are particularly preferred when the total amount of solids in the solution or suspension is close to 20% by weight, such as less than 40% by weight, in particular less than 30% or less than 25% by weight. When the solution or suspension has a solids content as low as this, the density of the particles, whilst sufficient for the purposes of the invention, can desirably be increased further by use of the above-described excipient compositions. However, if the solids content is above 30% by weight or more preferably above 40% by weight, the particles produced will be sufficiently dense, so that the extra density obtained by using the preferred excipients in the ratios described above is less important.

Any suitable spray freeze-drying technique can be used (for example the methods described by Mumenthaler et al., Int. J. Pharmaceutics (1991) 72, pages 97-110 and Maa et al., Phar. Res. (1999) Vol. 16, page 249) may be used to carry out the spray freeze-drying step.

A typical spray freeze-drying technique involves atomising the aqueous solution or suspension into a liquified gas, which is generally under stirring. The liquified gas can be liquid argon, liquid nitrogen, liquid oxygen or any other gas that results in the immediate freezing of the atomised droplets of the aqueous solution or suspension. Preferably the liquified gas is an inert liquified gas such as liquid nitrogen.

The liquified gas containing the frozen droplets of the aqueous solution or suspension is then freeze-dried. It is not contacted with an organic solvent such as methanol, ethanol, ethyl ether, acetone, pentane, P-pentane, methylene chloride, chloroform or ethyl acetate. Drying is not therefore conducted according to the procedures described in U.S. Pat. No. 5,019,400.

Typically, the liquified gas containing the frozen droplets is transferred into a lyophiliser for freeze-drying. The liquified gas containing the frozen droplets is usually poured into a metal tray and introduced into the lyophiliser. The frozen droplets are freeze-dried in the tray. The liquid nitrogen evaporates and the frozen water contained in the droplets is removed by sublimation. The resulting particles are collected. They can be washed as desired.

In more detail, the liquified gas containing frozen droplets of the atomized solution or suspension is held at reduced temperature, for example from about −60° C. to −40° C. Typically, that is followed by two-stage vacuum drying preferably under a pressure of from about 20 to 500 mT (2.666 to 66.65 Pa). The first drying stage is normally performed at a reduced temperature such as from about −50° C. to 0° C., for a period of about 4 to 24 hours. Frozen water is removed by ice sublimation. In the second drying stage, drying is normally performed at a higher temperature such as from about 5 to 30C at a lower pressure, preferably less than 100 mT down to about 10 mT, for a period of about 5 to 24 hours. The precise spray freeze-drying conditions used may be selected according to the desired properties of the particles to be produced. Thus, the temperatures, pressures and other conditions may be varied as desired.

Preferably, the nozzle used to atomise the solution or suspension is an ultrasonic nozzle. This has the advantage of being a mild process which generates little stress to the biomolecules which are frequently used as therapeutic agents in the present invention. In addition, use of an ultrasonic nozzle eliminates the need for pressurized gas to assist the liquid feed which, in turn helps increase the yield of the process. The predominant variable for control of droplet size in an ultrasonic nozzle system is the nozzle frequency, although surface tension, viscosity and density of the liquid feed are additional variables that can be manipulated to control droplet size. Thus, for example, smaller particles may be produced by increasing the nozzle frequency and vice versa. When using the ultrasonic nozzle system. am accurate. low-pulse feed pump can be used to delivery the liquid feed, wherein such pumps are particularly well suited when operating at low feed rates (e.g., about 3 to 5 ml per minute) normally associated with laboratory-scale particle production. It has been found that atomization proceeds well at about 1 to 2 Watts above the “critical power” level of the low-pulse pump system. In some drying cycles, it has been found that operation at about 2.9 to 3.1 Watts allows for the most efficient atomization, however the exact operating conditions will also depend upon the liquid characteristics of the feed (viscosity, density, total solids content, surface tension, etc.).

A dual spray freeze-drying process may also be used. This process is particularly useful when the pharmaceutical agent is a protein having a low water solubility. This dual process comprises spray freeze-drying the liquid protein to form a dry powder. This powder is then reconstituted in water to provide a suspension having the desired solid content and spray freeze-dried for a second time.

The spray freeze-dried particles that are obtained according to the invention can be collected, washed and dried. The dried particles can then be sieved to obtained particles of the desired size.

### Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

Hardening in a Non-Solvent

In this Example, primary particles are placed in a non-solvent (to both the particles and the plasticizer) to which small amounts of plasticizer are then added. The particles are continually agitated by use of a stirrer or impeller to prevent agglomeration. At this point a vacuum is applied to draw off the plasticizer after the particles have collapsed. Optionally, the dispersion may be warmed to raise the temperature to above the Tg of the particles. In another embodiment, the pressure of the suspension is increased to reduce the size of the particles.

### EXAMPLE 2

Fluidized-Bed Hardening

This example employs a fluidized bed to help disperse and keep separate the particles while solvent/plasticizer is applied and removed. A volatile solvent/plasticizer can be applied by spray or vapor and removed by using the fluidized bed as a dryer by altering the temperature and changing the recycle of air or inert gas. Alternatively the hardened particles can be collected in a non-solvent for the particles but a solvent or weak solvent for the plasticizer and collected by filtration. In this case the plasticizer can be non-volatile. With prudent choice of plasticizer, or a plasticizer in a solvent the plasticizer may be left behind after the hardening process, for example a solution of PVP in water may be added to a powder in minimal amounts and the water dried away leaving behind the PVP in the hardened particle. Likewise, the plasticizing solvent may contain a polymer which surface-coats as the particle is sprayed so that the drug release properties, surface quality and hardness are all modified in a single process step.

### EXAMPLE 3

Pre-Formulation for Hardening/Annealing

In this example a formulation is chosen in the primary particle formation process specifically to provide the capability of annealing or hardening in a second step. A small amount of plasticizer is included in the formulation during the primary formation step, for example spray freeze-drying. After the primary, larger-than-desired, not-sufficiently-dense or strong particles are harvested, they are placed in a non-solvent or a fluidized bed and the temperature elevated to collapse and harden the particles to optimal size, strength and density for powder injection. Likewise materials may be added to the powder to facilitate heating by absorption of infrared or microwave energy or to promote collapse, for example temperature sensitive hydrogels in an expanded state from the primary particle formation process.

### EXAMPLE 4

Two Step Production of Insulin in Carboxymethyl Cellulose

A hard, dense particulate form of insulin in a carboxymethyl cellulose carrier is prepared as follows:

An aqueous solution containing 4% by weight carboxymethyl cellulose is prepared. Insulin (0.04% by weight, basis solution) is added to the solution. This -material is fed to a spray drier. The spray drier air temperature is set at 120° C. The liquid flow and air flow are adjusted to yield a product which is free flowing and particulate in nature. One would expect this product to have an average particle size of from about 20 to about 70 μm depending upon the dimensions of the drier, the atomization conditions used, and the solids content of the aqueous solution. These parameters are readily available to the skilled artisan upon reading the instant specification, and general spray drying techniques can be found, for example, in the comprehensive text by Keith Masters, “Spray Drying Handbook,” (1985) Longman Scientific & Technical publ., John Wiley & Sons co-publs., New York, N.Y. These particles would also be expected to be relatively porous and soft with a typical specific gravity of 0.6 and a residual water content of about 5% w (basis particles). At this water level, the particles (about 10% w insulin, 5% w water and 94% carboxymethyl cellulose) exhibit a glass transition temperature within about 10-20% of the glass tansition temperature of the pure carboxymethyl cellulose.

These particles are collected and then suspended in cyclohexane nonsolvent and heated to about 81° C. Water is added to the mixture in the amount of 8% weight, basis particles. This water, being insoluble in the cyclohexane nonsolvent but being capable of associating with the generally hydrophilic carboxymethyl cellulose particles, preferentially adds to the particles to yield a water content of about 12% w. This lowers the glass transition temperature of the particles within about 30-40% of the glass transition temperature of the pure carboxymethyl cellulose, which is a temperature below the temperature at which the particles are being maintained. The particles are held in suspension at this temperature for about one hour. This causes the particles to collapse and/or shrink in size, thereby increasing the particle strength and density. The resulting particles would have an average diameter decrease of about 10-20% at most, which can provide a density increase of up to 100%.

The suspending process is halted. Bulk amounts of cyclohexane are decanted off of the particles. The particles are tumbled in a rotary drier under vacuum and under mild heating (e.g., about 40° C.). This drying is continued until the cyclohexane is removed and the residual water in the particles is reduced from about 12% w to about 2 to 4% weight. This provides particles with a glass transition temperature of about 50° C., which obviously is above the temperatures to which the particles typically would be exposed during storage and use.

The particles are loaded into a PowderJect® particle injector system (PowderJect Pharmaceuticals PLC, Oxford UK). Because of their durability and density imparted by the above-described process, they are suitably efficiently delivered to a patient using this system with minimal degradation during the injection process.

### EXAMPLE 5

Modified Two Step Production of Growth Hormone in Carboxylated Starch

A hard, dense particulate form of bovine growth hormone in a carboxylated starch carrier is prepared as follows:

Am aqueous solution is prepared containing 6% by weight carboxylmethyl starch. Bovine growth hormone (BGH) (0.01% by weight, basis solution) is added to the solution. This material is fed to a spray drier. The spray drier air temperature is set at 110° C. The liquid flow and air flow are adjusted to yield a product which is free flowing and particulate in nature. One would expect this product to have an average particle size of from about 20 to about 70 μm depending upon the dimensions of the drier, the atomization conditions used, and the solids content of the aqueous solution. These particles would also be expected to be relatively porous and soft with a typical specific gravity of 0.6 and a residual water content of about 5% w (basis particles). At this water level, the particles (about 0.17% w BGH, 5% w water and 94% carboxylmethyl starch) exhibit a glass transition temperature within about 10-20% of the glass transition temperature of the pure carboxymethyl starch, e.g., about 110° C.

These particles are not collected. Instead they are held in the spray drier in suspension, fluidized in air, at a temperature of about 50° C. Moisture is added to the suspending air flow so that the particles add water to a water level of about 8-10% by weight. This lowers the glass transition temperature of the particles to below 110° C. They are maintained at about 60° C. for about 30 minutes. This causes the particles to collapse and/or shrink in size, thereby increasing particle strength and density. The resulting particles will thus have an average diameter of about 40 μm and a specific gravity of about 1.2.

Dry air is fed into the drier to remove residual water and lower the particle's water level back to 2-3%. This causes the glass transition temperature of the particles to rise to within about 5% of the glass transition temperature of the carboxylmethyl starch carrier, which obviously is above the temperatures to which the particles typically would be exposed during storage and use.

The particles are loaded into a PowderJect® particle injector system (PowderJect Pharmaceuticals PLC, Oxford UK). Because of their durability and density imparted by the above-described process, they are suitably efficiently delivered to a patient using this system with minimal degradation during the injection process.

### EXAMPLE 6

Single Step Production of Growth Hormone in Carboxylated Starch

A hard, dense particulate form of bovine growth hormone in a carboxylmethyl starch carrier is prepared as follows:

An aqueous solution is prepared containing 6% by weight carboxylated starch. Bovine growth hormone (BGH) (0.0117, by weight, basis solution) is added to the solution. This material is fed to a spray drier. The spray drier air temperature is set at 110° C. The liquid flow and dry air flow are adjusted until a product is formed which is substantially free flowing and particulate in nature. One would expect this product to have an average particle size of about 60 μm, again dependent upon the dimensions of the drier, the atomization conditions used, and the solids content of the aqueous solution. These particles would also be expected to be relatively porous and soft with a typical specific gravity of 0.6 the flow of dry air is halted and moist air is recycled to maintain a residual water content of about 12% w (basis particles). At this water level, the particles (about 0.17% w BGH, 12% w water and 87% carboxylated starch) exhibit a glass transition temperature of within about 10-20% of the glass transition temperature of the pure carboxymethyl starch, e.g., about 110° C.

These particles are not collected. Instead they are held in the spray drier in suspension at about 60° C. and in 70% relative humidity. They are maintained at these conditions for about 30 minutes. This causes the particles to collapse and/or shrink in size, thereby increasing particle strength and density. The resulting particles will thus have an average diameter of about 40 μm and a specific gravity of about 1.2.

Dry air is fed into the drier to remove residual water and lower the particles' water level back to 2-3%. This causes the glass transition temperature of the particles to rise to about 125° C., which obviously is above the temperatures to which the particles typically would be exposed during storage and use.

The particles are loaded into a PowderJect(particle injector system (PowderJect Pharmaceuticals PLC, Oxford UK). Because of their durability and density imparted by the above-described process, they are suitably efficiently delivered to a patient using this system with minimal degradation during the injection process.

Accordingly, novel hard, dense particles are formed, and methods of generating these particles have been described. Although preferred embodiments of the subject invention have been described in some detail, it is understood that obvious variations can be made without departing from the spirit and the scope of the invention as defined by the appended claims.

What is claimed is:

1. A process for preparing solid particles of a pharmaceutical agent, comprising the steps of:
(a) forming first particles of a first size, said first particles comprising
an admixture of a pharmaceutical agent and a macromolecular carrier, said carrier capable of existing as a solid and said admixture having a first glass transition temperature, and
admixed plasticizer, said plasticizer lowering the first glass transition temperature of the admixture to a second glass transition temperature which is below the first glass transition temperature;
(b) maintaining said first particles at a temperature above the second glass transition temperature for a time period adequate to cause the first particles to shrink and/or collapse to second particles of a second size, which second size is smaller than the first size;
(c) removing plasticizer from the second particles to yield modified second particles thereby raising the glass transition temperature from said second glass transition temperature to a third glass transition temperature which is higher than second glass transition temperature;
(d) recovering the modified second particles; and
(e) storing said modified second particles at a temperature below the third glass transition temperature as the desired solid particles of the pharmaceutical agent.

2. The process of claim 1, wherein said macromolecular carrier comprises a naturally-occurring macromolecular material.

3. The process of claim 1, wherein said macromolecular carrier comprises a polysaccharide.

4. The process of claim 1, wherein said macromolecular carrier comprises a polypeptide.

5. The process of claim 1, wherein said macromolecular carrier comprises a synthetic polymer.

6. The process of claim 1, wherein said pharmaceutical agent is a pharmaceutically active protein.

7. The process of claim 1, wherein said pharmaceutical agent is a small molecule drug.

8. The process of claim 1, wherein said pharmaceutical agent is a polynucleotide.

9. The process of claim 1, wherein the particles are formed in step (a) by spray-drying.

10. The process of claim 1, wherein the particles are formed in step (a) by spray freeze-drying.

11. The process of claim 1, wherein the particles are formed in step (a) by grinding or milling.

12. The process of claim 1, wherein the plasticizer is removed in step (c) by evaporation and/or washing.

13. The process of claim 1, wherein the modified second particles are stored in step (e) at ambient temperature.

14. A process for preparing solid particles of a pharmaceutical agent, comprising the steps of:
(a) forming an admixture comprising a pharmaceutical agent and macromolecular carrier, said admixture having a first glass transition temperature;
(b) forming particles of a first size of said admixture;
(c) adding plasticizer to said particles of a first size to yield plasticized particles, said plasticizer lowering the glass transition temperature of the admixture to a second glass transition temperature below said first glass transition temperature;
(d) maintaining said plasticized particles at a temperature above said second glass transition temperature for a time period adequate to cause said plasticized particles to shrink and/or collapse to particles of a second size which second size is smaller than the first size;
(e) removing plasticizer from the particles of a second size to yield modified particles of a second size thereby raising the glass transition temperature from said second glass transition temperature to a third glass transition temperature which is higher than second glass transition temperature;
(f) recovering the modified particles of a second size; and
(g) storing said modified particles of the second size at a temperature below the third glass transition temperature as the desired solid particles of the pharmaceutical agent.

15. The process of claim 14, wherein said macromolecular carrier comprises a naturally-occurring macromolecular material.

16. The process of claim 14, wherein said macromolecular carrier comprises a polysaccharide.

17. The process of claim 14, wherein said macromolecular carrier comprises a polypeptide.

18. The process of claim 14, wherein said macromolecular carrier comprises a synthetic polymer.

19. The process of claim 14, wherein said pharmaceutical agent is a pharmaceutically active protein.

20. The process of claim 14, wherein said pharmaceutical agent is a small molecule drug.

21. The process of claim 14, wherein said pharmaceutical agent is a polynucleotide.

22. The process of claim 14, wherein the particles are formed in step (b) by spray drying.

23. The process of claim 14, wherein said particles are formed in step (b) by spray freeze-drying.

24. The process of claim 14, wherein the particles are formed in step (b) by grinding or milling.

25. The process of claim 14, wherein the plasticizer is removed in step (e) by evaporation and/or washing.

26. The process of claim 14, wherein the modified particles of the second size are stored in step (g) at ambient temperature.

27. A process for preparing solid particles of a pharmaceutical agent, comprising the steps of:
(a) forming an admixture comprising a pharmaceutical agent, a macromolecular carrier and plasticizer;
said pharmaceutical agent and said macromolecular carrier having a first glass transition temperature when separately admixed;
and said pharmaceutical agent, said macromolecular carrier and said plasticizer having a second glass transition temperature when together admixed, said second glass transition temperature being below said first glass transition temperature;
(b) forming first particles of a first size of said admixture;
(c) maintaining said first particles of a first size at a temperature above said second glass transition temperature for a time period adequate to cause said first particles to shrink and/or collapse to particles of a second size which second size is smaller than the first size;
(d) removing plasticizer from the particles of a second size to yield modified particles of a second size thereby raising the glass transition temperature from said second glass transition temperature to a third glass transition temperature which is higher than second glass transition temperature;
(e) recovering the modified particles of a second size; and
(f) storing said modified particles of the second size at a temperature below the third glass transition temperature as the desired solid particles of the pharmaceutical agent.

28. The process of claim 27, wherein said macromolecular carrier comprises a naturally-occurring macromolecular material.

29. The process of claim 27, wherein said macromolecular carrier comprises a polysaccharide.

30. The process of claim 27, wherein said macromolecular carrier comprises a polypeptide.

31. The process of claim 27, wherein said macromolecular carrier comprises a synthetic polymer.

32. The process of claim 27, wherein said pharmaceutical agent is a pharmaceutically active protein.

33. The process of claim 27, wherein said pharmaceutical agent is a small molecule drug.

34. The process of claim 27, wherein said pharmaceutical agent is a polynucleotide.

35. The process of claim 27, wherein the particles are formed in step (b) by spray drying.

36. The process of claim 27, wherein the particles are formed in step (b) by spray freeze-drying.

37. The process of claim 27, wherein the particles are formed in step (b) by grinding or milling.

38. The process of claim 27, wherein the plasticizer is removed in step (d) by evaporation and/or washing.

39. The process of claim 27, wherein the modified particles of the second size are stored in step (f) at ambient temperature.

40. Solid particles of a pharmaceutical agent prepared by a process comprising the steps of:
(a) forming first particles of a first size, said first particles comprising
an admixture of a pharmaceutical agent and a macromolecular carrier, said carrier capable of existing as a solid and said admixture having a first glass transition temperature, and
admixed plasticizer, said plasticizer lowering the first glass transition temperature of the admixture to a second glass transition temperature which is below the first glass transition temperature;
(b) maintaining said first particles at a temperature above the second glass transition temperature for a time period adequate to cause the first particles to shrink and/or collapse to second particles of a second size, which second size is smaller than the first size;
(c) removing plasticizer from the second particles to yield modified second particles thereby raising the glass transition temperature from said second glass transition temperature to a third glass transition temperature which is higher than second glass transition temperature;
(d) recovering the modified second particles; and
(e) storing said modified second particles at a temperature below the third glass transition temperature as the desired solid particles of the pharmaceutical agent.

41. Solid particles according to claim 40 prepared by a process comprising the steps of:
(a) forming an admixture comprising a pharmaceutical agent and macromolecular carrier, said admixture having a first glass transition temperature;
(b) forming particles of a first size of said admixture;
(c) adding plasticizer to said particles of a first size to yield plasticized particles, said plasticizer lowering the glass transition temperature of the admixture to a second glass transition temperature below said first glass transition temperature;
(d) maintaining said plasticized particles at a temperature above said second glass transition temperature for a time period adequate to cause said plasticized particles to shrink and/or collapse to particles of a second size which second size is smaller than the first size;
(e) removing plasticizer from the particles of a second size to yield modified particles of a second size thereby raising the glass transition temperature from said second glass transition temperature to a third glass transition temperature which is higher than second glass transition temperature;
(f) recovering the modified particles of a second size; and
(g) storing said modified particles of the second size at a temperature below the third glass transition temperature as the desired solid particles of the pharmaceutical agent.

42. Solid particles according to claim 40 prepared by a process comprising the steps of:
(a) forming an admixture comprising a pharmaceutical agent, a macromolecular carrier and plasticizer; said pharmaceutical agent and said macromolecular carrier having a first glass transition temperature when separately admixed; and said pharmaceutical agent, said macromolecular carrier and said plasticizer having a second glass transition temperature when together admixed, said second glass transition temperature being below said first glass transition temperature;
(b) forming first particles of a first size of said admixture;
(c) maintaining said first particles of a first size at a temperature above said second glass transition temperature for a time period adequate to cause said first particles to shrink and/or collapse to particles of a second size which second size is smaller than the first size;
(d) removing plasticizer from the particles of a second size to yield modified particles of a second size thereby raising the glass transition temperature from said second glass transition temperature to a third glass transition temperature which is higher than second glass transition temperature;
(e) recovering the modified particles of a second size; and
(f) storing said modified particles of the second size at a temperature below the third glass transition temperature as the desired solid particles of the pharmaceutical agent.
